SEQUENCE LISTING (58 sequences)

SEQ ID NO: 1 - protein, 12 aa, Artificial Sequence
  Hair-binding Peptide
TPPELLHGDPRS

SEQ ID NO: 2 - protein, 7 aa, Artificial Sequence
  Hair-binding Peptide
NTSQLST

SEQ ID NO: 3 - protein, 8 aa, Artificial Sequence
  Hair-binding Peptide
RTNAADHP

SEQ ID NO: 4 - protein, 12 aa, Artificial Sequence
  Hair-binding Peptide
RTNAADHPAAVT

SEQ ID NO: 5 - protein, 12 aa, Artificial Sequence
  Hair-binding Peptide
IPWWNIRAPLNA

SEQ ID NO: 6 - protein, 7 aa, Artificial Sequence
  Hair-binding Peptide
DLTLPFH

SEQ ID NO: 7 - protein, 12 aa, Artificial Sequence
  Empirically Generated Hair and Skin-Binding Peptide
KRGRHKRPKRHK

SEQ ID NO: 8 - protein, 7 aa, Artificial Sequence
  Empirically Generated Hair and Skin-Binding Peptide
RLLRLLR

SEQ ID NO: 9 - protein, 12 aa, Artificial Sequence
  Empirically Generated Hair and Skin-Binding Peptide
HKPRGGRKKALH

SEQ ID NO: 10 - protein, 18 aa, Artificial Sequence
  Empirically Generated Hair and Skin-Binding Peptide
KPRPPHGKKHRPKHRPKK

SEQ ID NO: 11 - protein, 18 aa, Artificial Sequence
  Empirically Generated Hair and Skin-Binding Peptide
RGRPKKGHGKRPGHRARK

SEQ ID NO: 12 - protein, 67 aa, Artificial Sequence
  Multiple Hair-Binding Peptide
PNTSQLSTGGGRTNAADHPKCGGGNTSQLSTGGGRTNAADHPKCGGGNTSQLSTGGGRTNAADHPKC

SEQ ID NO: 13 - protein, 55 aa, Artificial Sequence
  Multiple Hair-Binding Peptide
PRTNAADHPAAVTGGGCGGGRTNAADHPAAVTGGGCGGGRTNAADHPAAVTGGGC

SEQ ID NO: 14 - protein, 50 aa, Artificial Sequence
  Multiple Hair-Binding Peptide
PRTNAADHPAAVTGGGCGGGIPWWNIRAPLNAGGGCGGGDLTLPFHGGGC

SEQ ID NO: 15 - protein, 82 aa, Artificial Sequence
  Multiple Hair-Binding Peptide
PRTNAADHPGGGTPPELLHGDPRSKCGGGRTNAADHPGGGTPPELLHGDPRSKCGGGRTNAADHPGGGTPPELLHGDPRSKC

SEQ ID NO: 16 - protein, 82 aa, Artificial Sequence
  Multiple Hair-Binding Peptide
PTPPTNVLMLATKGGGRTNAADHPKCGGGTPPTNVLMLATKGGGRTNAADHPKCGGGTPPTNVLMLATKGGGRTNAADHPKC

SEQ ID NO: 17 - protein, 82 aa, Artificial Sequence
  Multiple Hair-Binding Peptide
PRTNAADHPGGGTPPTNVLMLATKKCGGGRTNAADHPGGGTPPTNVLMLATKKCGGGRTNAADHPGGGTPPTNVLMLATKKC

SEQ ID NO: 18 - protein, 13 aa, Artificial Sequence
  Hair-binding peptide haveing a cysteine residue added to the C-terminus
TPPELLHGDPRSC

SEQ ID NO: 19 - protein, 12 aa, Artificial Sequence
  Hair-Binding Peptide
EQISGSLVAAPW

SEQ ID NO: 20 - protein, 12 aa, Artificial Sequence
  Hair-Binding Peptide
TDMQAPTKSYSN

SEQ ID NO: 21 - protein, 11 aa, Artificial Sequence
  Hair-Binding Peptide
LPRIANTWSPS

SEQ ID NO: 22 - protein, 12 aa, Artificial Sequence
  Hair-Binding Peptide
LDTSFPPVPFHA

SEQ ID NO: 23 - protein, 12 aa, Artificial Sequence
  Hair-Binding Peptide
TPPTNVLMLATK

SEQ ID NO: 24 - protein, 15 aa, Artificial Sequence
  Hair-Binding Peptide
STLHKYKSQDPTPHH

SEQ ID NO: 25 - protein, 12 aa, Artificial Sequence
  Hair-Binding Peptide
GMPAMHWIHPFA

SEQ ID NO: 26 - protein, 15 aa, Artificial Sequence
  Hair-Binding Peptide
HDHKNQKETHQRHAA

SEQ ID NO: 27 - protein, 20 aa, Artificial Sequence
  Hair-Binding Peptide
HNHMQERYTDPQHSPSVNGL

SEQ ID NO: 28 - protein, 20 aa, Artificial Sequence
  Hair-Binding Peptide
TAEIQSSKNPNPHPQRSWTN

SEQ ID NO: 29 - protein, 8 aa, Artificial Sequence
  Caspase 3 Cleavage site
LESGDEVD

SEQ ID NO: 30 - protein, 37 aa, Artificial Sequence
  Peptide Spacer
TSTSKASTTTTSSKTTTTSSKTTTTTSKTSTTSSSST

SEQ ID NO: 31 - protein, 22 aa, Artificial Sequence
  Peptide Spacer
GQGGYGGLGSQGAGRGGLGGQG

SEQ ID NO: 32 - protein, 10 aa, Artificial Sequence
  Peptide Spacer
GPGGYGPGQQ

SEQ ID NO: 33 - protein, 60 aa, Artificial Sequence
  Multiple Hair-Binding Peptide
PGIPWWNIRAPLNAGAGIPWWNIRAPLNAGGSGPGSGGNTSQLSTGGGNTSQLSTGGPKK

SEQ ID NO: 34 - DNA, 203 bp, Artificial Sequence
  Gene Encoding Multiple Hair-Binding Peptide HC77643
ggatccgaccctggtatcccgtggtggaacattcgcgcacctctgaatgctggtgctggtattccgtggtggaacatccgtgctcctctgaacgcgggtggctccggtccgggctccggtggcaacacgagccaactgagcaccggtggtggcaacacttcccagctgtccaccggcggtccgaaaaagtaataaggcgcgcc

SEQ ID NO: 35 - DNA, 5388 bp, Artificial Sequence
  Plasmid pKSIC4-HC77623
agatctcgatcccgcgaaattaatacgactcactatagggagaccacaacggtttccctctagaaataattttgtttaactttaagaaggagatatacatatgcataccccagaacacatcaccgccgtggtacagcgctttgtggctgcgctcaatgccggcgatctggacggcatcgtcgcgctgtttgccgatgacgccacggtggaagagcccgtgggttccgagcccaggtccggtacggctgcgtgtcgtgagttttacgccaactcgctcaaactgcctttggcggtggagctgacgcaggagtgccgcgcggtcgccaacgaagcggccttcgctttcaccgtcagcttcgagtatcagggccgcaagaccgtagttgcgccctgtgatcactttcgcttcaatggcgccggcaaggtggtgagcatccgcgccttgtttggcgagaagaatattcacgcatgccagggatccgatccgactccgccgacgaatgtactgatgctggcaaccaaaggcggtggtacgcattccacgcacaaccatggcagcccgcgccacacgaatgctgacgcaggcaatccgggcggcggcaccccaccaaccaatgtcctgatgctggctactaaaggcggcggcacgcattctacccacaaccatggtagcccgcgccatactaatgcagatgccggcaacccgggcggtggtaccccgccaaccaacgttctgatgctggcgacgaaaggtggcggtacccattccacgcataatcatggcagccctcgccacaccaacgctgatgctggtaatcctggtggcggtaagaagaaataataaggcgcgccgacccagctttcttgtacaaagtggttgattcgaggctgctaacaaagcccgaaaggaagctgagttggctgctgccaccgctgagcaataactagcataaccccttggggcctctaaacgggtcttgaggggttttttgctgaaaggaggaactatatccggatatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattaaagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgatcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcg

SEQ ID NO: 36 - protein, 3 aa, Artificial Sequence
  Peptide Spacer
GGG

SEQ ID NO: 37 - protein, 7 aa, Artificial Sequence
  Peptide Spacer
GGGCGGG

SEQ ID NO: 38 - protein, 4 aa, Artificial Sequence
  Peptide Spacer
GGGC

SEQ ID NO: 39 - protein, 3 aa, Artificial Sequence
  Peptide Spacer
GAG

SEQ ID NO: 40 - protein, 9 aa, Artificial Sequence
  Peptide Spacer
GGSGPGSGG

SEQ ID NO: 41 - protein, 5 aa, Artificial Sequence
  Peptide Spacer
GGPKK

SEQ ID NO: 42 - protein, 20 aa, artificial sequence
  Hair-binding peptide
HINKTNPHQGNHHSEKTQRQ

SEQ ID NO: 43 - protein, 15 aa, artificial sequence
  Hair-binding peptide
HAHKNQKETHQRHAA

SEQ ID NO: 44 - protein, 15 aa, artificial sequence
  Hair-binding peptide
HEHKNQKETHQRHAA

SEQ ID NO: 45 - protein, 20 aa, artificial sequence
  Hair-binding peptide
HNHMQERYTEPQHSPSVNGL

SEQ ID NO: 46 - protein, 20 aa, artificial sequence
  Hair-binding peptide
THSTHNHGSPRHTNADAGNP

SEQ ID NO: 47 - protein, 17 aa, artificial sequence
  Hair-binding peptide
THSTHNHGSPRHTNADA

SEQ ID NO: 48 - protein, 15 aa, artificial sequence
  Hair-binding peptide
HHGTHHNATKQKNHV

SEQ ID NO: 49 - protein, 13 aa, artificial sequence
  Hair-binding peptide
STLHKYKSQDPTP

SEQ ID NO: 50 - protein, 20 aa, artificial sequence
  Hair-binding peptide
GSCVDTHKADSCVANNGPAT

SEQ ID NO: 51 - protein, 20 aa, artificial sequence
  Hair-binding peptide
AQSQLPDKHSGLHERAPQRY

SEQ ID NO: 52 - protein, 20 aa, artificial sequence
  Hair-binding peptide
AQSQLPAKHSGLHERAPQRY

SEQ ID NO: 53 - protein, 20 aa, artificial sequence
  Hair-binding peptide
AQSQLPEKHSGLHERAPQRY

SEQ ID NO: 54 - protein, 20 aa, artificial sequence
  Hair-binding peptide
TDMMHNHSDNSPPHRRSPRN

SEQ ID NO: 55 - protein, 20 aa, artificial sequence
  Hair-binding peptide
TPPELAHTPHHLAQTRLTDR

SEQ ID NO: 56 - protein, 12 aa, artificial sequence
  Hair-binding peptide
RLLRLLRLLRLL

SEQ ID NO: 57 - protein, 12 aa, artificial sequence
  Hair-binding peptide
TPPELLHGEPRS

SEQ ID NO: 58 - protein, 12 aa, artificial sequence
  Hair-binding peptide
TPPELLHGAPRS

(19) United States
(12) Patent Application Publication
(10) Pub. No.: US 2008/0175798 A1
(43) Pub. Date: Jul. 24, 2008

(54) PEPTIDE-BASED HAIR PROTECTANTS

(76) Inventors: WILLIAM A. BECK, Middletown, DE (US); John P. O'Brien, Oxford, PA (US); Hong Wang, Kennett Square, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(21) Appl. No.: 11/939,583

(22) Filed: Nov. 14, 2007

Related U.S. Application Data
(60) Provisional application No. 60/869,363, filed on Dec. 11, 2006.

(51) Int. Cl. A61K 8/02; C07K 2/00; A61K 8/64

(52) U.S. Cl. 424/47; 530/300; 424/70.9

(57) ABSTRACT

Peptide-based hair protectants, formed by coupling at least one hair-binding peptide with at least one sunscreen agent, are described. The hair-binding peptide portion of the peptide-based hair protectant binds to hair with high affinity, thus keeping the sunscreen agent attached to the hair for long lasting protection. Hair care compositions comprising the peptide-based hair protectants are also described.

This application claims the benefit of U.S. Provisional Application No. 60/869,363 filed Dec. 11, 2006.

### FIELD OF THE INVENTION

The invention relates to the field of personal care products. More specifically, the invention relates to peptide-based hair protectants formed by coupling a hair-binding peptide with a sunscreen agent.

### BACKGROUND OF THE INVENTION

The harmful effects of ultraviolet radiation from sunlight to the skin are well documented. Ultraviolet radiation, both UVB (ultraviolet radiation of wavelengths between 290 to 320 nanometers) and UVA (ultraviolet radiation in the wavelength range of 320 to 400 nanometers), also causes damage to hair. Prolonged exposure to ultraviolet radiation may result in physical and chemical changes that cause weakened, dry and brittle hair. Additionally, the color of hair, both natural and dyed, can be altered by the bleaching effect of ultraviolet radiation.

Damage to the hair from sunlight can be controlled by utilizing sunscreen agents that absorb or scatter ultraviolet radiation form the sun. Hair care products comprising sunscreen agents are known in the art (see for example, Ciaudelli et al., U.S. Pat. No. 4,567,038; Smith et al. U.S. Pat. No. 4,786,493; Luther et al. U.S. Pat. No. 6,090,370; and Djerassi et al. U.S. Patent Application Publication No. 2002/0131939). However, these sunscreen agents have only a short-term effect because they are not strongly attached to the hair. A more durable, long-lasting hair protectant, which protects the hair from the ultraviolet radiation of the sun, would represent an advance in the art.

In order to improve the durability of hair and skin care products, peptide-based hair conditioners, hair colorants, and other benefit agents have been developed (Huang et al., co-pending and commonly owned U.S. Pat. No. 7,220,405, and U.S. Patent Application Publication No. 2005/0226839). The peptide-based benefit agents are prepared by coupling a specific peptide sequence that has a high binding affinity to hair or skin with a benefit agent. The peptide portion binds to the hair or skin, thereby strongly attaching the benefit agent. Additionally, peptide-based inorganic sunscreens comprising a skin-binding peptide coupled to an inorganic metal oxide sunscreen agent (Buseman-Williams et al., co-pending and commonly owned U.S. Patent Application Publication No. 2005/0249682) and peptide-based organic sunscreens comprising a skin-binding peptide coupled to an organic sunscreen agent (Lowe et al., co-pending and commonly owned U.S. Patent Application Publication No. 2007-0110686) have been reported. However, hair protectants formed by coupling a hair-binding peptide to a sunscreen agent have not been described.

Peptides having a binding affinity to hair and skin have been identified using phage display screening techniques (Huang et al., supra; Estell et al. WO01/79479; Murray et al., U.S. Patent Application Publication No. 2002/0098524; Janssen et al., U.S. Patent Application Publication No. 2003/0152976; and Janssen et al., WO04/048399). Additionally, empirically generated hair and skin-binding peptides that are based on positively charged amino acids have been reported (Rothe et., WO 2004/000257).

In view of the above, a need exists for hair protectants that provide improved durability for long lasting effects and are easy and inexpensive to prepare.

Applicants have addressed the stated need by designing peptide-based hair protectants formed by coupling hair-binding peptides, which bind to hair with high affinity, to sunscreen agents to give hair protectants that provide long lasting protection.

### SUMMARY OF THE INVENTION

The invention provides peptide-based hair protectants formed by coupling at least one hair-binding peptide with at least one sunscreen agent. Accordingly, in one embodiment the invention provides a peptide-based hair protectant having the general structure:

(HBPₘ)ₙ-(SCA)_{y}

or

[(HBP)ₚ—S_{q}]ₙ-(SCA)_{y}

wherein

a) HBP is a hair-binding peptide;

b) SCA is a sunscreen agent;

c) m ranges from 1 to about 100;

d) n ranges from 1 to about 100;

e) y ranges from 1 to about 100;

f) S is a spacer;

g) p ranges from 1 to 10; and

h) q ranges from 1 to about 100.

In another embodiment, the invention provides a hair care composition comprising an effective amount of at least one peptide-based hair protectant.

The invention also provides methods for forming a protective layer of a peptide-based hair protectant on hair comprising applying the hair care composition of the invention to the hair and allowing the formation of the protective layer.

In another embodiment, the invention provides a method for forming a protective layer on hair comprising the steps of:
(a) providing a hair care composition comprising at least one peptide-based hair protectant selected from the group consisting of:

(HBPₘ)ₙ-(SCA)_{y}; and i)

[(HBP)ₚ-S_{q}]ₙ-(SCA)_{y} ii)
wherein
   1) HBP is a hair-binding peptide;
   2) SCA is a sunscreen agent;
   3) n ranges from 1 to about 100;
   4) S is a spacer;
   5) m ranges from 1 to about 100;
   6) p ranges from 1 to about 10;
   7) q ranges from 1 to about 100; and
   8) y ranges from 1 to about 100;
and wherein the hair-binding peptide is selected by a method comprising the steps of:
   (A) providing a combinatorial library of DNA associated peptides;
   (B) contacting the library of (A) with a hair sample to form a reaction mixture comprising DNA associated peptide-hair complexes;
   (C) isolating the DNA associated peptide-hair complexes of (B);
   (D) amplifying the DNA encoding the peptide portion of the DNA associated peptide-hair complexes of (C), wherein the peptide is a hair-binding peptide; and
   (E) sequencing the amplified DNA of (D) encoding a hair-binding peptide, wherein the hair-binding peptide is identified; and
(b) applying the hair care composition of (a) to hair and allowing the formation of said protective layer.

### BRIEF DESCRIPTION OF FIGURES AND SEQUENCE DESCRIPTIONS

The various embodiments of the invention can be more fully understood from the following detailed description, the figure and the accompanying sequence descriptions, which form a part of this application.

FIG. 1 is a plasmid map of the vector pKSIC4-HC77623, described in Example 2.

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37C.F.R. §1.822.

SEQ ID NOs: 1-11 and 18-28 are the amino acid sequences of hair-binding peptides.

SEQ ID NO: 29 is the amino acid sequence of the protease Caspase 3 cleavage site.

SEQ ID NOs: 30, 31, 32, and 36-41 are the amino acid sequences of peptide spacers.

SEQ ID NOs: 12-17, and 33 are the amino acid sequences of multi-block hair-binding peptides.

SEQ ID NO: 34 is the nucleotide sequence of the gene used to prepare the multi-block hair-binding peptide sequence given as SEQ ID NO:33.

SEQ ID NO: 35 is the nucleotide sequence of plasmid pKSIC4—HC77623, which is described in Example 2.

SEQ ID NOs: 42-58 are the amino acid sequences of hair-binding peptides.

### DETAILED DESCRIPTION OF THE INVENTION

The present invention provides long lasting hair protectants formed by coupling at least one hair-binding peptide to at least one sunscreen agent. The peptide-based hair protectants may be used in hair care products to protect the hair from damage caused by ultraviolet radiation from the sun. The hair care compositions of the invention provide improved water resistance due to the affinity of the hair-binding peptide to the hair, thereby eliminating or reducing the need for reapplication of the composition after exposure of the hair to water.

The following definitions are used herein and should be referred to for interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

“HBP” means hair-binding peptide.

“SCA” means sunscreen agent.

“S” means spacer. “Spacer” or “linker” will be used interchangeably and will refer to an entity that links the hair-binding peptide with the sunscreen agent. The spacer or linker may be comprised of amino acids or may be a chemical linker.

The term “peptide” refers to two or more amino acids joined to each other by peptide bonds or modified peptide bonds.

The term “hair-binding peptide” refers to peptide sequences that bind with high affinity to hair. In one embodiment, the hair-binding peptide is selected from the group consisting of SEQ ID NOs: 1-28, 33, and 42-58.

In another embodiment, the hair-binding peptides are from about 7 amino acids to about 50 amino acids, more preferably, from about 7 amino acids to about 25 amino acids, and most preferably from about 7 to about 20 amino acids in length.

In a further embodiment, the hair-binding peptides also include multi-block hair-binding peptides (for example, see SEQ ID NOs: 12-17 and 33). Multi-block hair-binding peptides are hair-binding peptides comprising two or more individual hair-binding peptide segments, where the individual hair-binding peptide segments may or may not have the same sequences, optionally separated by one or more spacers. The spacer may be a chemical spacer molecule, a peptide spacer, or a combination of an organic spacer molecule and a peptide spacer. In a preferred embodiment, the spacer is a peptide spacer. In the context of this disclosure, a “multi-block” hair-binding peptide also refers as a “multiple” hair-binding peptide.”

The term “DNA associated peptide” or “nucleic acid associated peptide” refers to a peptide having associated with it an identifying nucleic acid component. In the case of ribosome display or mRNA display, the DNA associated peptide may include peptides associated with their mRNA progenitor (i.e. an identifying nucleic acid component) that can be reverse translated into cDNA. In a phage display system, peptides are displayed on the surface of the phage while the DNA encoding the peptides is contained within the attached glycoprotein coat of the phage. The association of the coding DNA within the phage may be used to facilitate the amplification of the coding region for the identification of the peptide.

The term “DNA associated peptide-hair complex” refers to a complex between hair and a DNA associated peptide wherein the peptide is bound to the hair via a binding site on the peptide.

The terms “coupling” and “coupled” as used herein refer to any chemical association and includes both covalent and non-covalent interactions.

The term “stringency” as it is applied to the selection of the hair-binding peptides of the present invention, refers to the concentration of the eluting agent used to elute peptides from the hair. Higher concentrations of the eluting agent provide more stringent conditions.

The term “MB₅₀” refers to the concentration of the binding peptide that gives a signal that is 50% of the maximum signal obtained in an ELISA-based binding assay as described herein. The MB₅₀ provides an indication of the strength of the binding interaction or affinity of the components of the complex. The lower the value of MB₅₀, the stronger the interaction of the peptide with its corresponding substrate.

The term “binding affinity” refers to the strength of the interaction of a binding peptide with its respective substrate. The binding affinity is defined herein in terms of the MB₅₀ value, determined in an ELISA-based binding assay. In one embodiment, “high affinity” is defined as an MB₅₀ value of no more than 10⁻⁴ M, preferably no more than 10⁻⁵ M, more preferably no more than 10⁻⁶ M, even more preferably no more than 10⁻⁷ M, and most preferably less than or equal to 10⁻⁸ M.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any (or as defined herein) | Xaa | X |

“Gene” refers to a nucleic acid fragment that expresses a specific protein, optionally including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “transformation” refers to the transfer of a nucleic acid fragment into a host organism, resulting in genetically stable inheritance. Host organisms containing the transformed nucleic acid fragments are referred to as “transgenic” or “recombinant” or “transformed” organisms.

The term “host cell” refers to a cell which has been transformed or transfected, or is capable of transformation or transfection by an exogenous polynucleotide sequence.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

“PCR” or “polymerase chain reaction” is a technique used for the amplification of specific DNA segments (U.S. Pat. Nos. 4,683,195 and 4,800,159).

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

The invention provides peptide-based hair protectants formed by coupling at least one hair-binding peptide to at least one sunscreen agent. The hair-binding peptide may be identified using combinatorial methods, such as phage display, bacterial display, yeast display, ribosome display, or mRNA display. Alternatively, hair-binding peptide sequences may be generated empirically by designing peptides that comprise certain amino acids such as positively charged amino acids, which facilitates the peptides binding to the negative charged surface of hair via electrostatic interaction, as described by Rothe et al. (WO 2004/000257). The hair-binding peptide is coupled to the sunscreen agent, either directly or via an optional spacer, using covalent or non-covalent attachment. The peptide-based hair protectants may be used in hair care products to protect the hair from damage caused by ultraviolet radiation from the sun.

### Hair-Binding Peptides

Hair-binding peptides (HBP), as defined herein, are peptide sequences that bind with high affinity to hair. In one embodiment, the sequence of the hair-binding peptide is selected from the group consisting of SEQ ID NOs: 1-28, 33, and 42-58. In a preferred embodiment, the sequence of hair-binding peptide is selected from the group consisting of SEQ ID NOs: 1-11, 18-28, and 42-58. In another embodiment, the hair-binding peptides of the invention are from about 7 amino acids to about 50 amino acids, more preferably, from about 7 amino acids to about 25 amino acids, and most preferably from about 7 to about 20 amino acids in length. Suitable hair-binding peptides may be selected using methods that are well known in the art or may be generated empirically. In another embodiment, the hair-binding peptide is a multi-block hair-binding peptide comprising two or more hair-binding peptides (i.e. HPB, wherein m, n or p is greater than 1), optionally separated by a peptide spacer (S). The individual hair-binding peptides within a multi-block hair-binding peptide may be the same or different. In another embodiment, the sequence of the multi-block hair-binding peptide is selected from the group consisting of SEQ ID NOs: 12-17 and 33.

The hair-binding peptides may be generated randomly and then selected against a specific hair sample based upon their binding affinity for the hair sample, as described by Huang et al. in co-pending and commonly owned U.S. Pat. No. 7,220,405 and U.S. Patent Application Publication No. 2005-0226839. The generation of random libraries of peptides is well known and may be accomplished by a variety of techniques including, bacterial display (Kemp, D. J.; Proc. Natl. Acad. Sci. USA 78(7): 4520-4524 (1981); yeast display (Chien et al., Proc Natl Acad Sci USA 88(21): 9578-82 (1991)), combinatorial solid phase peptide synthesis (U.S. Pat. No. 5,449,754; U.S. Pat. No. 5,480,971; U.S. Pat. No. 5,585,275 and U.S. Pat. No. 5,639,603), phage display technology (U.S. Pat. No. 5,223,409; U.S. Pat. No. 5,403,484; U.S. Pat. No. 5,571,698; and U.S. Pat. No. 5,837,500), ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Exemplary methods used to generate such biological peptide libraries are described in Dani, M., J. of Receptor & Signal Transduction Res., 21(4):447-468 (2001), Sidhu et al., Methods in Enzymology 328:333-363 (2000), and Phage Display of Peptides and Proteins, A Laboratory Manual, Brian K. Kay, Jill Winter, and John McCafferty, eds.; Academic Press, NY, 1996. Additionally, phage display libraries are available commercially from companies such as New England Biolabs (Beverly, Mass.).

### Phage Display

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide (phenotype) and the DNA encoding it (genotype) allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

The present hair-binding peptides may be identified using the following process. After a suitable library of DNA associated peptides has been generated using phage display, the library of DNA associated peptides is dissolved in a suitable solution for contacting a hair sample. In one embodiment, the library of DNA associated peptides is dissolved in a buffered aqueous saline solution containing a surfactant. A suitable solution is Tris-buffered saline (TBS) with 0.5% TWEEN® 20. The library of DNA associated peptides is contacted with an appropriate amount of hair sample to form a reaction mixture. Human hair samples are available commercially, for example from International Hair Importers and Products (Bellerose, N.Y.), in different colors, such as brown, black, red, and blond, and in various types, such as African-American, Caucasian, and Asian. Additionally, the hair samples may be treated, for example, using hydrogen peroxide to obtain bleached hair or subjected to a dye treatment to obtain “dyed-hair” (see co-pending and co-owned U.S. Provisional Patent Application No. 60/972,312; herein incorporated by reference). The mixture may be agitated by any means in order to increase the mass transfer rate of the DNA associated peptides to the hair surface, thereby shortening the time required to attain maximum binding. The time required to attain maximum binding varies depending on a number of factors, such as size of the hair sample, the concentration of the peptide library, and the agitation rate. The time required can be determined readily, by one skilled in the art, using routine experimentation. Typically, the contact time is 10 minutes to one hour. To remove undesired DNA associated peptides that bind to a non-target, such as skin or plastic, the library of DNA associated peptides may optionally be contacted with the non-target either prior to or simultaneously with contacting the hair sample.

Upon contact, a number of the randomly generated DNA associated peptides will bind to the hair to form a DNA associated peptide-hair complex. Unbound peptide may be removed by washing. After all unbound material is removed, DNA associated peptides having varying degrees of binding affinities for hair may be fractionated by selected washings using washing solutions having varying stringencies. As the stringency of the washing solution increases, the bond strength between the peptide and hair in the remaining DNA associated peptide-hair complex increases.

A number of substances may be used to vary the stringency of the washing solution in the peptide selection process including, but not limited to, acids (pH 1.5-3.0), bases (pH 10-12.5), salts of high concentrations such as MgCl₂ (3-5 M) and LiCl (5-10 M), ethylene glycol (25-50%), dioxane (5-20%), thiocyanate (1-5 M), guanidine (2-5 M), urea (2-8 M), and surfactants of various concentrations such as SDS (sodium dodecyl sulfate), DOC (sodium deoxycholate), Nonidet P-40, Triton X-100, TWEEN® 20, wherein TWEEN® 20 is more typical. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred.

It will be appreciated that DNA associated peptides having increasing binding affinities for hair may be eluted by repeating the selection process using washing solutions with increasing stringencies.

The DNA associated peptide-hair complexes may then be contacted with an eluting agent for a period of time, typically, 1 to 30 minutes, to dissociate the DNA associated peptides from the hair; however, a portion of the DNA associated peptides may still remain bound to the hair after this treatment. Optionally, the DNA associated peptide-hair complexes are transferred to a new container before contacting with the eluting agent. The eluting agent may be any known eluting agent including, but not limited to, acids (pH 1.5-3.0), bases (pH 10-12.5), salts of high concentrations such as MgCl₂ (3-5 M) and LiCl (5-10 M), water; ethylene glycol (25-50%), dioxane (5-20%), thiocyanate (1-5 M), guanidine (2-5 M), and urea (2-8 M), wherein treatment with an acid is more typical. If the eluting agent used is an acid or a base, then, a neutralization buffer is added after the elution step to adjust the pH of the eluent to the neutral range. Any suitable neutralization buffer may be used, wherein 1 M Tris-HCl at pH 9.2 is an example of a buffer that may be used after an acidic eluting agent.

The eluted DNA associated peptides or the remaining bound DNA associated peptides, or both the eluted DNA associated peptides and the remaining bound DNA associated peptides are then amplified using methods known in the art. For example, the eluted DNA associated peptides and the remaining bound DNA associated peptides may be amplified by infecting/transfecting a bacterial host cell, such as E. coli ER2738, as described by Huang et al. in U.S. Pat. No. 7,220,405. The infected host cells are grown in a suitable growth medium, such as LB (Luria-Bertani) medium, and this culture is spread onto agar, containing a suitable growth medium, such as LB medium with IPTG (isopropyl β-D-thiogalactopyranoside) and S-GAL™ (3,4-cyclohexenoesculetin-β-D-galactopyranoside). After growth, the plaques are picked for DNA isolation and sequencing to identify the hair-binding peptide sequences. Alternatively, the eluted DNA associated peptides and the remaining bound DNA associated peptides may be amplified using a nucleic acid amplification method, such as the polymerase chain reaction (PCR), to amplify the DNA comprising a hair-binding peptide coding region. In that approach, PCR is carried out on the DNA encoding the eluted DNA associated peptides and/or the remaining bound DNA associated peptides using the appropriate primers, as described by Janssen et al. in U.S. Patent Application Publication No. 2003/0152976, which is incorporated herein by reference.

In one embodiment, the eluted DNA associated peptides and the remaining bound DNA associated peptides are amplified by infecting a bacterial host cell as described above, the amplified DNA associated peptides are contacted with a fresh hair sample, and the entire process described above is repeated one or more times to obtain a population that is enriched in hair-binding DNA associated peptides (provided that the peptides were generated by phage display). After the desired number of biopanning cycles, the amplified DNA associated peptide sequences are determined using standard DNA sequencing techniques that are well known in the art to identify the hair-binding peptide sequences. Hair-binding peptide sequences identified using this method include, but are not limited to, SEQ ID NOs: 1-6 (Table 1). Additional hair binding peptides identified by phage display also include SEQ ID NOs: 42-58.

Additionally, shampoo-resistant hair-binding peptides may be selected using a modified biopanning method as described by O'Brien et al. in co-pending and commonly owned U.S. Patent Application Publication No. 2006/0073111. Similarly, hair conditioner-resistant hair-binding peptides may be identified using the method described by Wang et al. (co-pending and commonly owned U.S. Patent Application Publication No. 2007-0196305). In those methods, either suspended the initial library of phage peptides in the matrix of interest (i.e., a shampoo matrix or a hair conditioner matrix) for contacting with the substrate (i.e. hair), or contact the phage-peptide substrate complex with the matrix of interest after the complex is formed, as described above, by contacting the substrate (i.e. hair) with the library of phage peptides. The biopanning method is then conducted as described above. The shampoo matrix or hair conditioner matrix may be a full strength commercial product or a dilution thereof. Examples of shampoo-resistant and hair conditioner-resistant hair-binding peptides include, but are limited to, hair-binding sequences, given as SEQ ID NOs: 23-28 (see Table 1). Hair-binding peptide sequences may also be determined using the method described by Lowe in co-pending and commonly owned U.S. Patent Application Publication No. 2006-0286047. That method provides a means for determining the sequence of a peptide binding motif having affinity for a particular substrate, for example hair. First, a population of binding peptides for the substrate of interest is identified by biopanning using a combinatorial method, such as phage display. Rather than using many rounds of biopanning to identify specific binding peptide sequences and then using standard pattern recognition techniques to identify binding motifs, as is conventionally done in the art, the method requires only a few rounds of biopanning. The sequences in the population of binding peptides, which are generated by biopanning, are analyzed by identifying subsequences of 2, 3, 4, and 5 amino acid residues that occur more frequently than expected by random chance. The identified subsequences are then matched head to tail to give peptide motifs with substrate binding properties. This procedure may be repeated many times to generate long peptide sequences.

Alternatively, hair-binding peptide sequences may be generated empirically by designing peptides that comprise positively charged amino acids, which can bind to hair via electrostatic interaction, as described by Rothe et al. (WO 2004/000257). The empirically generated hair-binding peptides have between about 7 amino acids to about 50 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred, where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Examples of empirically generated hair-binding peptides include, but are not limited to SEQ ID NOs: 7-11 (Table 1).

The hair-binding peptide may further comprise at least one cysteine or lysine residue on at least one of the C-terminal end or the N-terminal end of the hair-binding peptide sequence to facilitate coupling with the sunscreen agent, as described below. Examples of a hair-binding peptide comprising a cysteine residue at the C-terminal end include SEQ ID NOs: 12-18. Examples of a hair-binding peptide comprising a lysine residue at the C-terminal end include SEQ ID NOs: 7,10,11, 23 and 33.

TABLE 1
Examples of Hair-Binding Peptide Sequences

| | SEQ | |
|---|---|---|
| Body Surface | ID NO: | Sequence |
| Hair | 1 | TPPELLHGDPRS |
| (Shampoo Resistant) | | |
| Hair | 2 | NTSQLST |
| (Shampoo Resistant) | | |
| Hair | 3 | RTNAADHP |
| Hair | 4 | RTNAADHPAAVT |
| Hair | 5 | IPWWNIRAPLNA |
| Hair | 6 | DLTLPFH |
| Hair and Skin | 7 | KRGRHKRPKRHK |
| (empirical) | | |
| Hair and Skin | 8 | RLLRLLR |
| (empirical) | | |
| Hair and Skin | 9 | HKPRGGRKKALH |
| (empirical) | | |
| Hair and Skin | 10 | KPRPPHGKKHRPKHRPKK |
| (empirical) | | |
| Hair and Skin | 11 | RGRPKKGHGKRPGHRARK |
| (empirical) | | |
| Hair (Multiple) | 12 | P-NTSQLST (hair-binding peptide)-GGG |
| | | (spacer)-RTNAADHPKC (hair-binding |
| | | peptide)-GGG (spacer)-NTSQLST (hair- |
| | | binding peptide)-GGG (spacer)- |
| | | RTNAADHPKC (hair-binding peptide)- |
| | | GGG (spacer)-NTSQLST (hair-binding |
| | | peptide)-GGG (spacer)-RTNAADHPKC |
| | | (hair-binding peptide) |
| Hair (Multiple) | 13 | P-RTNAADHPAAVT (hair-binding |
| | | peptide)-GGGCGGG (spacer)- |
| | | RTNAADHPAAVT (hair-binding peptide)- |
| | | GGGCGGG (spacer)-RTNAADHPAAVT |
| | | (hair-binding peptide)-GGGC (spacer) |
| Hair (Multiple) | 14 | P-RTNAADHPAAVT (hair-binding |
| | | peptide)-GGGCGGG (spacer)- |
| | | IPWWNIRAPLNA (hair-binding peptide)- |
| | | GGGCGGG (spacer)-DLTLPFH (hair- |
| | | binding peptide)-GGGC (spacer) |
| Hair (Multiple) | 15 | P-RTNAADHP (hair-binding peptide)- |
| | | GGG (spacer)-TPPELLHGDPRSKC |
| | | (hair-binding peptide)-GGG (spacer)- |
| | | RTNAADHP (hair-binding peptide)-GGG |
| | | (spacer)-TPPELLHGDPRSKC (hair- |
| | | binding peptide)-GGG (spacer)- |
| | | RTNAADHP (hair-binding peptide)-GGG |
| | | (spacer)-TPPELLHGDPRSKC (hair- |
| | | binding peptide) |
| Hair (Multiple) | 16 | P-TPPTNVLMLATK (hair-binding |
| | | peptide)-GGG (spacer)-RTNAADHPKC |
| | | (hair-binding peptide)-GGG (spacer)- |
| | | TPPTNVLMLATK (hair-binding peptide)- |
| | | GGG (spacer)-RTNAADHPKC (hair- |
| | | binding peptide)-GGG (spacer)- |
| | | TPPTNVLMLATK (hair-binding peptide)- |
| | | GGG (spacer)-RTNAADHPKC (hair- |
| | | binding peptide) |
| Hair (Multiple) | 17 | P-RTNAADHP (hair-binding peptide)- |
| | | GGG (spacer)-TPPTNVLMLATKKC |
| | | (hair-binding peptide)-GGG (spacer)- |
| | | RTNAADHP (hair-binding peptide)-GGG |
| | | (spacer)-TPPTNVLMLATKKC (hair- |
| | | binding peptide) GGG (spacer)- |
| | | RTNAADHP (hair-binding peptide) GGG |
| | | (spacer)-TPPTNVLMLATKKC (hair- |
| | | binding peptide) |
| Hair (Multiple) | 33 | PG (Spacer)-IPWWNIRAPLNA (hair- |
| | | binding peptide)- GAG (spacer)- |
| | | IPWWNIRAPLNA (hair-binding peptide)- |
| | | GGSGPGSGG (spacer)- |
| | | NTSQLST (hair-binding peptide)- |
| | | GGG (spacer)- |
| | | NTSQLST (hair-binding peptide)-GGPKK |
| | | (spacer) |
| Hair (with cysteine | 18 | TPPELLHGDPRSC |
| at C-terminus) | | |
| Hair | 19 | EQISGSLVAAPW |
| Hair | 20 | TDMQAPTKSYSN |
| Hair | 21 | ALPRIANTWSPS |
| Hair | 22 | LDTSFPPVPFHA |
| Hair | 23 | TPPTNVLMLATK |
| (Shampoo Resistant) | | |
| Hair | 24 | STLHKYKSQDPTPHH |
| (Conditioner Resistant) | | |
| Hair (Shampoo | 25 | GMPAMHWIHPFA |
| and Conditioner | | |
| Resistant) | | |
| Hair (Shampoo | 26 | HDHKNQKETHQRHAA |
| and Conditioner | | |
| Resistant) | | |
| Hair (Shampoo | 27 | HNHMQERYTDPQHSPSVNGL |
| and Conditioner | | |
| Resistant) | | |
| Hair (Shampoo | 28 | TAEIQSSKNPNPHPQRSWTN |
| and Conditioner | | |
| Resistant) | | |

### Production of Hair-Binding Peptides

The hair-binding peptides of the present invention may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the peptides of the present invention may be prepared using recombinant DNA and molecular cloning techniques. Genes encoding the hair-binding peptides may be produced in heterologous host cells, particularly in the cells of microbial hosts, as described by Huang et al. (U.S. Pat. No. 7,220,405), and as exemplified in Example 2, below. The peptides when prepared by recombinant DNA and molecular cloning techniques may further comprise a proline (P) residue at the N-terminus and optionally an aspartic acid (D) residue at the C-terminus. These additional residues result from the use of DP cleavage sites to separate the desired peptide sequence from peptide tags, used to promote inclusion body formation, and between tandem repeats of the peptide sequences

### Peptide-Based Hair Protectants

The peptide-based hair protectants of the present invention are formed by coupling at least one hair-binding peptide (HBP) with at least one sunscreen agent (SCA). The hair-binding peptide part of the hair protectant binds strongly to the hair, thus keeping the sunscreen agent attached to the hair for long-lasting protection. Suitable hair-binding peptides include, but are not limited to SEQ ID NOs: 1-28, 33, and 42-58. Any known hair-binding peptide sequence may be used including, but not limited to SEQ ID NO: 19-22, as described by Janssen et al. in U.S. Patent Application Publication No. 2003/0152976 and WO 04048399. It may also be desirable to link two or more hair-binding peptides together, either directly or through a spacer, to enhance the interaction with the hair. Non-limiting examples of these multiple hair-binding peptides (“multi-block” hair-binding peptides) are given as SEQ ID NOs: 12-17 and 33. Methods to prepare the multiple hair-binding peptides and suitable spacers are described below.

Sunscreen agents are well known in the art, and include both inorganic sunscreen agents and organic sunscreen agents. Inorganic sunscreen agents function by reflecting, scattering and/or absorbing ultraviolet radiation. Suitable inorganic sunscreen agents for use in the present invention include, but are not limited to, inorganic pigments and metal oxides including oxides of titanium (e.g., SunSmart available from Cognis Corp., Monheim, Germany), zinc, cerium, and iron. A preferred inorganic sunscreen is titanium dioxide nanoparticles. Suitable titanium dioxide nanoparticles are described in U.S. Pat. Nos. 5,451,390; 5,672,330; and 5,762,914. Titanium dioxide P25 is an example of a suitable commercial product available from Degussa (Parsippany, N.J.). Other commercial suppliers of titanium dioxide nanoparticles include Kemira (Helsinki, Finland), Sachtleben (Duisburg, Germany) and Tayca (Osaka, Japan).

The titanium dioxide nanoparticles typically have an average particle size diameter of less than 100 nanometers (nm) as determined by dynamic light scattering which measures the particle size distribution of particles in liquid suspension. The particles are typically agglomerates which may range from about 3 nm to about 6000 nm. Any process known in the art can be used to prepare such particles. The process may involve vapor phase oxidation of titanium halides or solution precipitation from soluble titanium complexes, provided that titanium dioxide nanoparticles are produced.

A preferred process to prepare titanium dioxide nanoparticles is by injecting oxygen and titanium halide, preferably titanium tetrachloride, into a high-temperature reaction zone, typically ranging from 400° C. to 2000° C. Under the high temperature conditions present in the reaction zone, nanoparticles of titanium dioxide are formed having high surface area and a narrow size distribution. The energy source in the reactor may be any heating source such as a plasma torch.

Organic sunscreen agents are organic chemicals that absorb or scatter ultraviolet light of wavelengths between 290 and 400 nm. Organic sunscreen agents are well known in the art (see for example, Woodin et al., U.S. Pat. No. 5,219,558, which is incorporated herein by reference, in particular column 3, line 35 to column 4, line 23). Suitable examples of organic sunscreen agents include, but are not limited to, para-aminobenzoic acid (PABA), ethyl para-aminobenzoate, amyl para-aminobenzoate, octyl para-aminobenzoate, ethylhexyl dimethyl para-aminobenzoate (Padimate O), ethylene glycol salicylate, phenyl salicylate, octyl salicylate, benzyl salicylate, butylphenyl salicylate, homomethyl salicylate (Homosalate), ethylhexyl salicylate (Octisalate), triethanolamine salicylate (Trolamine salicylate), benzyl cinnamate, 2-ethoxyethyl para-methoxycinnamate (such as PARSOL® available from Givaudan-Roure Co., Vernier, Switzerland), ethylhexyl methoxycinnamate (Octinoxate), octyl para-methoxycinnamate, glyceryl mono(2-ethylhexanoate) di-para-methoxycinnamate, isopropyl para-methoxycinnamate, urocanic acid, ethyl urocanate, hydroxymethoxybenzophenone (Benzophenone-3), hydroxymethoxybenzophenonesulfonic acid (Benzophenone-4) and salts thereof, dihydroxymethoxybenzophenone (Benzophenone-8), sodium dihydroxymethoxybenzophenonedisulfonate, dihydroxybenzophenone, tetrahydroxybenzophenone, 4-tert-butyl-4′-methoxydibenzoylmethane (Avobenzone), phenylbenzimidazole sulfonic acid (Ensulizole), 2,4,6-trianilino-p-(carbo-2′-ethylhexyl-1′-oxy)-1,3,5-triazine, octocrylene, menthyl anthranilate (Meradimate), cinnamic acid, 2-(2-hydroxy-5-methylphenyl)benzotriazole, and derivatives thereof. The sunscreen agent may also be an organic polymer that scatters ultraviolet radiation, thereby enhancing the absorption of the radiation by other sunscreen agents. An example of this type of sunscreen agent is SUNSPHERES™ Polymer, available from Rohm and Haas Co. (Philadelphia, Pa.).

The peptide-based hair protectants of the present invention are prepared by coupling at least one specific hair-binding peptide to at least one sunscreen agent, either directly or via an optional spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based hair protectant may be prepared by mixing the peptide with the sunscreen agent and the optional spacer (if used) and allowing sufficient time for the interaction to occur. The uncoupled materials may be separated from the resulting peptide-based hair protectant using methods known in the art, for example, extractions or chromatographic methods.

The peptide-based hair protectants of the invention may also be prepared by covalently attaching at least one specific hair-binding peptide to at least one sunscreen agent, either directly or through a spacer. Any known peptide or protein conjugation chemistry may be used to form the peptide-based hair protectants of the present invention. Conjugation chemistries are well known in the art (see for example, Hermanson, Bioconjugate Techniques, Academic Press, New York (1996)). Suitable coupling agents include, but are not limited to, carbodiimide coupling agents, acid chlorides, isocyanates, epoxides, maleimides, and other functional coupling reagents that are reactive toward terminal amine and/or carboxylic acid groups, and sulfhydryl groups on the peptides. Additionally, it may be necessary to protect reactive amine or carboxylic acid groups on the peptide to produce the desired structure for the peptide-based hair protectant. The use of protecting groups for amino acids, such as t-butyloxycarbonyl (t-Boc), are well known in the art (see for example Stewart et al., supra; Bodanszky, supra; and Pennington et al., supra). In some cases it may be necessary to introduce reactive groups, such as carboxylic acid, alcohol, amine, isocyanate, or aldehyde groups to the sunscreen agent for coupling to the hair-binding peptide. These modifications may be done using routine chemistry such as oxidation, reduction, phosgenation, and the like, which is well known in the art.

It may also be desirable to couple the hair-binding peptide to the sunscreen agent via a spacer. The spacer serves to separate the sunscreen agent from the peptide to ensure that the agent does not interfere with the binding of the peptide to the hair. The spacer may be any of a variety of molecules, such as alkyl chains, phenyl compounds, ethylene glycol, amides, esters and the like. The spacer may be covalently attached to the peptide and the sunscreen agent using any of the coupling chemistries described above. In order to facilitate incorporation of the spacer, a bifunctional coupling agent that contains a spacer and reactive groups at both ends for coupling to the peptide and the organic sunscreen agent may be used.

The spacer may be any of a variety of molecules, such as alkyl chains, phenyl compounds, ethylene glycol, amides, esters and the like. Preferred spacers are hydrophilic and have a chain length from 1 to about 100 atoms, more preferably, from 2 to about 30 atoms. Examples of preferred spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains. The spacer may be covalently attached to the hair-binding peptide and the sunscreen agent sequences using any of the coupling chemistries described above.

Additionally, the spacer may be a peptide comprising any amino acid and mixtures thereof. The preferred peptide spacers are comprised of the amino acids proline, lysine, glycine, alanine, serine, and mixtures thereof. In addition, the peptide spacer may comprise a specific enzyme cleavage site, such as the protease Caspase 3 site, given as SEQ ID NO: 29, which allows for the enzymatic removal of the organic sunscreen agent from the hair. The peptide spacer may be from 2 to about 50 amino acids, preferably from 2 to about 20 amino acids in length. Exemplary peptide spacers comprise amino acid sequences including, but are not limited to, SEQ ID NOs: 30, 31, 32, and 36-41. These peptide spacers may be linked to the hair-binding peptide by any method known in the art. For example, the entire binding peptide-peptide spacer diblock (i.e. [(HBP)ₚ-S_{q}]ₙ) may be prepared using the standard peptide synthesis methods described above. In addition, the hair-binding peptide and the peptide spacer may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid groups on the peptides. Alternatively, the entire hair-binding peptide-peptide spacer diblock (i.e. [(HBP)ₚ-S_{q}]ₙ) may be prepared using the recombinant DNA and molecular cloning techniques described above. The spacer may also be a combination of a peptide spacer and an organic spacer molecule, which may be prepared using the methods described above.

It may also be desirable to have multiple hair-binding peptides coupled to the sunscreen agent to enhance the interaction between the peptide-based hair protectant and the hair. Either multiple copies of the same hair-binding peptide or a combination of different hair-binding peptides may be used. Typically, 1 to about 100 hair-binding peptides can be coupled to a sunscreen agent. Additionally, multiple peptide sequences may be linked together and attached to the organic sunscreen agent, as described above. Typically, up to about 100 hair-binding peptides may be linked together. Moreover, multiple sunscreen agents (SCA) may be coupled to the hair-binding peptide. Therefore, in one embodiment of the present invention, the peptide-based hair protectants are compositions consisting of a hair-binding peptide (HBP) and an sunscreen agent (SCA), having the general structure (HBPₘ)ₙ-(SCA)_{y}, where m, n and y independently range from 1 to about 100, preferably from 1 to about 10.

In another embodiment, the peptide-based hair protectants contain a spacer (S) separating the hair-binding peptide from the sunscreen agent, as described above. Multiple copies of the hair-binding peptide may be coupled to a single spacer molecule. Additionally, multiple copies of the peptides may be linked together via spacers and coupled to the sunscreen agent via a spacer. Moreover, multiple sunscreen agents (SCA) may be coupled to the spacer. In this embodiment, the peptide-based hair protectants are compositions consisting of a hair-binding peptide, a spacer, and a sunscreen agent, having the general structure [(HBP)ₚ-S_{q}]ₙ-(SCA)_{y}, where p ranges from 1 to about 10, preferably p is 1, and q, n, and y independently range from 1 to about 100, preferably q, n, and y independently range from 1 to about 10.

It should be understood that as used herein, HBP is a generic designation and is not meant to refer to a single hair-binding peptide. Where m, n or p as used above, is greater than 1, it is well within the scope of the invention to provide for the situation where a series of hair-binding peptides of different sequences may form a part of the composition. In addition, “S” is also a generic term and is not meant to refer to a single spacer. Where q or n, as used above, is greater than 1, it is well within the scope of the invention to provide for the situation where a number of different spacers may form a part of the composition. Similarly, “SCA” “is also a generic term and is not meant to refer to a single sunscreen agent. Where y, as used above, is greater than 1, it is well within the scope of the invention to provide for the situation where a number of different sunscreen agents may form a part of the composition. Additionally, it should be understood that these structures do not necessarily represent a covalent bond between the peptide, the sunscreen agent, and the optional spacer. As described above, the coupling interaction between the peptide, the sunscreen agent, and the optional spacer may be either covalent or non-covalent.

### Hair Care Compositions

The peptide-based hair protectants of the invention may be used in a variety of hair care compositions. Hair care compositions are herein defined as compositions for the treatment of hair including, but not limited to, shampoos, conditioners, rinses, lotions, aerosols, gels, mousses, and hair dyes.

The hair care compositions of the invention comprise an effective amount of at least one peptide-based hair protectant. An effective amount of a peptide-based hair protectant for use in a hair care composition is herein defined as a proportion of from about 0.01% to about 30%, preferably about 0.01% to about 10% by weight relative to the total weight of the composition. This proportion may vary as a function of the type of hair care composition. Additionally, the hair care composition may comprise a mixture of different peptide-based hair protectants. If a mixture of different peptide-based hair protectants is used in the composition, the total concentration of the peptide-based hair protectants is about 0.01% to about 30%, preferably about 0.01% to about 10% by weight relative to the total weight of the composition.

The hair care composition may comprise a cosmetically acceptable medium for hair care compositions, examples of which are described for example by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight, for the aqueous-alcoholic solutions. Additionally, the hair care compositions may contain one or more conventional cosmetic or dermatological additives, or adjuvants including, but not limited to antioxidants, preserving agents, fillers, surfactants, fragrances, thickeners, wetting agents, anionic polymers, nonionic polymers, amphoteric polymers, dyes and pigments.

In one embodiment, the hair care composition comprising the peptide-based hair protectants of the invention is a shampoo composition.

In another embodiment, the hair care composition comprising the peptide-based hair protectants of the invention is a hair conditioner composition.

### Methods for Treating Hair

In another embodiment, a method is provided for treating hair with the hair care compositions of the invention. Specifically, the present invention also comprises a method for forming a protective layer of peptide-based hair protectant on hair by applying one of the compositions described above comprising an effective amount of at least one peptide-based hair protectant to the hair and allowing the formation of the protective layer. The compositions of the present invention may be applied to the hair by various means including, but not limited to spraying, brushing, and applying by hand.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “g” means gram(s), “mg” means milligram(s), “mol” means mole(s), “mL” means milliliter(s), “L” means liter(s), “h” means hour(s), “nm” means nanometer(s), “μm” means micrometer(s), “wt %” means percent by weight, “vol %” means percent by volume, “qs” means as much as suffices, “MALDI mass spectrometry” means matrix-assisted, laser desorption ionization mass spectrometry, “EDTA” means ethylenediamine tetraacetate, “CFTA” means the Cosmetic, Toiletry and Fragrance Association, “OD₆₀₀” means the optical density measured at a wavelength of 600 nm, “rpm” means revolutions per minute, “atm” means atmosphere(s), “kPa” means kilopascals, “SLPM” means standard liter per minute, “psi” means pounds per square inch, “RCF” means relative centrifugal field.

### Example 1

### Preparation of a Peptide-Based Hair Protectant

The purpose of this Example was to prepare a peptide-based hair protectant by covalently coupling a hair-binding peptide to the sunscreen agent cinnamic acid functionalized with acyl chloride.

Acyl chloride functionalized cinnamic acid (3-phenyl-2-propenoyl chloride, CAS No. 102-92-1, obtained from Aldrich; Milwaukee, Wis.), 18 mg, was dissolved in 5 mL of 1-methyl-2-pyrrolidone (NMP) and added to a solution containing 64 mg of trifluoroacetate salt of an unprotected hair-binding peptide having a sequence as SEQ ID NO:26 (obtained from SynBioSci, Livermore, Calif.), dissolved in 10 mL of NMP containing triethylamine (50 mg). The resulting solution was stirred at room temperature for 96 h. After that time, the solvent was removed by evaporation, yielding 95 mg of an orange, waxy solid.

The crude product was analyzed by gas chromatography-MALDI mass spectrometry and found to exhibit product molecular weights consistent with covalent attachment of multiple cinnamic acid moieties to the peptide. Small amounts of peptide fragments not containing cinnamic acid moieties along with other unreacted peptide molecules were also present.

### Example 2

### Preparation of a Peptide-Based Hair Protectant

The purpose of this Example was to prepare a peptide-based hair protectant by covalently coupling a multi-block hair-binding peptide to the sunscreen agent cinnamic acid functionalized with acyl chloride. The multi-block hair-binding peptide was prepared using recombinant DNA and molecular cloning techniques.

### Biological Production of the Multi-Block Hair-Binding Peptide

The peptides were expressed in E. coli as inclusion bodies. Additional amino acid sequences (i.e., peptide tags) were fused to the multi-block hair-binding peptide sequence in order to promote inclusion body formation. Acid-labile Asp-Pro (DP) sequences were placed between the peptide tag and the multiple hair-binding peptide sequence to facilitate isolation of the multiple hair-binding peptide from the peptide tag.

### Construction of Production Strains

The sequences of the multi-block hair-binding peptide is given in Table 2. DNA sequences were designed to encode this peptide sequence using favorable codons for E. coli and to avoid sequence repeats and mRNA secondary structure. The gene DNA sequence was designed by DNA 2.0, Inc. (Menlo Park, Calif.) using proprietary software which is described by Gustafsson et al. (Trends in Biotechnol. 22(7):346-355 (2004)). The sequence encoding the amino acid sequence was followed by two termination codons and a recognition site for endonuclease Ascl. The GS amino acid sequence at the N-terminus was encoded by a recognition site for endonuclease BamHI (GGA/TCC). The DNA sequence is given by SEQ ID NO:34.

TABLE 2
Peptide Sequence and DNA Encoding Sequence of Multiple Hair-Binding peptide

| Multiple | | | Peptide | DNA |
|---|---|---|---|---|
| Hair-Binding | | | SEQ | SEQ |
| Peptide | Peptide Sequence | DNA Sequence* | ID NO: | ID NO: |
| HC77643 | PG (Spacer)-IPWWNIRAPLNA | GGATCCGACCCTGGTATCCCGTGGTGGAACA | 33 | 34 |
| | (hair-binding peptide)-GAG | TTCGCGCACCTCTGAATGCTGGTGCTGGTATT | | |
| | (spacer)-IPWWNIRAPLNA | CCGTGGTGGAACATCCGTGCTCCTCTGAACG | | |
| | (hair-binding peptide)- | CGGGTGGCTCCGGTCCGGGCTCCGGTGGCA | | |
| | GGSGPGSGG (spacer)-NTSQLST | ACACGAGCCAACTGAGCACCGGTGGTGGCA | | |
| | (hair-binding peptide)-GGG | ACACTTCCCAGCTGTCCACCGGCGGGTCCGAA | | |
| | (spacer)- NTSQLST (hair-binding | AAAGTAATAAGGCGCGCC | | |
| | peptide)-GGPKK (spacer) | | | |

*The coding sequence for the multi-block hair-binding peptide is underlined.

The gene was assembled from synthetic oligonucleotides and cloned into a standard plasmid cloning vector by DNA 2.0, Inc. The sequence was verified by DNA sequencing by DNA 2.0, Inc.

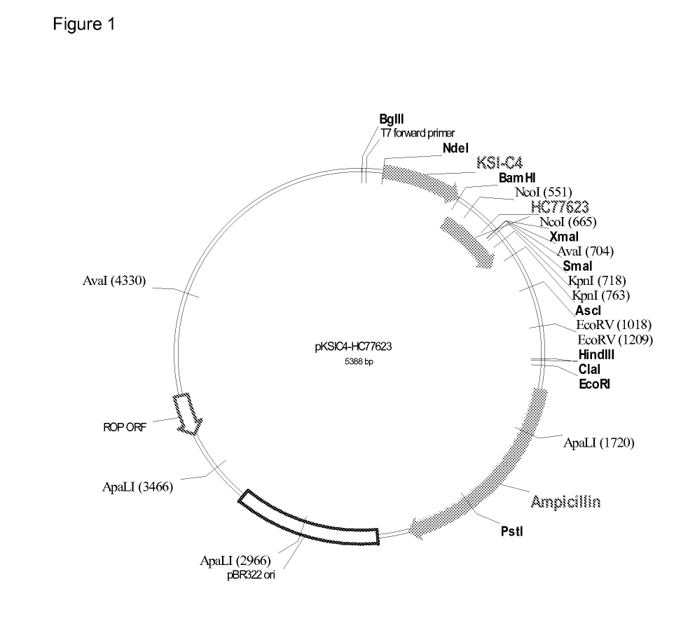

The synthetic gene was excised from the cloning vector with the endonuclease restriction enzymes BamHI and AscI and ligated into an expression vector using standard recombinant DNA methods. The vector pKSIC4-HC77623 was derived from the commercially available vector pDEST17 (Invitrogen, Carlsbad, Calif.). It includes sequences derived from the commercially available vector pET31b (Novagen, Madison, Wis.) that encode a fragment of the enzyme ketosteroid isomerase (KSI). The KSI fragment was included as a fusion partner to promote partition of the peptides into insoluble inclusion bodies in E. coli. The KSI-encoding sequence from pET31 b was modified using standard mutagenesis procedures (QuickChange II, Stratagene, La Jolla, Calif.) to include three additional Cys codons, in addition to the one Cys codon found in the wild type KSI sequence. The plasmid pKSIC4-HC77623, given by SEQ ID NO:35 and shown in FIG. 1, was constructed using standard recombinant DNA methods, which are well known to those skilled in the art.

The DNA sequence encoding the multiple hair-binding peptide (Table 2) was inserted into pKSIC4-HC77623 by substituting for sequences in the vector between the BamHI and AscI sites. Plasmid DNA containing the peptide encoding sequence and vector DNA was digested with endonuclease restriction enzymes BamHI and AscI, then the peptide-encoding sequence and vector DNA were mixed and ligated by phage T4 DNA ligase using standard DNA cloning procedures, which are well known to those skilled in the art. The correct construct, in which the sequence encoding the multiple hair-binding peptide was inserted into pKSIC4-HC77623, was identified by restriction analysis and verified by DNA sequencing, using standard methods.

In this construct, the sequence encoding the multiple hair-binding peptide was substituted for those encoding HC77623. The sequence was operably linked to the bacteriophage T7 gene 10 promoter and expressed as a fusion protein, fused with the variant KSI partner.

To test the expression of the multiple hair-binding peptide, the expression plasmid was transformed into the BL21-AI E. coli strain (Invitrogen, catalog no. C6070-03). To produce the recombinant fusion peptide, 50 mL of LB-ampicillin broth (10 g/L bacto-tryptone, 5 g/L bacto-yeast extract, 10 g/L NaCl, 100 mg/L ampicillin, pH 7.0) was inoculated with the transformed bacteria and the culture was shaken at 37° C. until the OD₆₀₀ reached 0.6. The expression was induced by adding 0.5 mL of 20 wt % L-arabinose to the culture and shaking was continued for another 4 h. Analysis of the cell protein by polyacrylamide gel electrophoresis demonstrated the production of the fusion peptides.

### Fermentation:

The recombinant E. coli strain, described above, was grown in a 6-L fermentation, which was run in batch mode initially, and then in fed-batch mode. The composition of the fermentation medium is given in Table 3. The pH of the fermentation medium was 6.7. The fermentation medium was sterilized by autoclaving, after which the following sterilized components were added: thiamine hydrochloride (4.5 mg/L), glucose (22.1 g/L), trace elements, see Table 4 (10 mL/L), ampicillin (100 mg/L), and inoculum (seed) (125 mL). The pH was adjusted as needed using ammonium hydroxide (20 vol %) or phosphoric acid (20 vol %). The added components were sterilized either by autoclaving or filtration.

TABLE 3
Composition of Fermentation Medium

| Component | Concentration | |
|---|---|---|
| KH₂PO₄ | 9 | g/L |
| (NH₄)₂HPO₄ | 4 | g/L |
| MgSO₄•7H₂O | 1.2 | g/L |
| Citric Acid | 1.7 | g/L |
| Yeast extract | 5.0 | g/L |
| Mazu DF 204 Antifoam | 0.1 | mL/L |

TABLE 4
Trace Elements

| Component | Concentration, mg/L |
|---|---|
| EDTA | 840 |
| CoCl₂•H₂O | 250 |
| MnCl₂•4H₂O | 1500 |
| CuCl₂•2H₂O | 150 |
| H₃BO₃ | 300 |
| Na₂MoO₄•2H₂O | 250 |
| Zn(CH₃COO)₂•H₂O | 1300 |
| Ferric citrate | 10000 |

The operating conditions for the fermentations are summarized in Table 5. The initial concentration of glucose was 22.1 g/L. When the initial residual glucose was depleted, a pre-scheduled, exponential glucose feed was initiated starting the fed-batch phase of the fermentation run. The glucose feed (see Tables 6 and 7) contained 500 g/L of glucose and was supplemented with 5 g/L of yeast extract. The components of the feed medium were sterilized either by autoclaving or filtration. The goal was to sustain a specific growth rate of 0.13 h⁻¹, assuming a yield coefficient (biomass to glucose) of 0.25 g/g, and to maintain the acetic acid levels in the fermentation vessel at very low values (i.e. less than 0.2 g/L). The glucose feed continued until the end of the run. Induction was initiated with a bolus of 2 g/L of L-arabinose at the selected time (i.e. 15 h of elapsed fermentation time). A bolus to deliver 5 g of yeast extract per liter of fermentation broth was added to the fermentation vessel at the following times: 1 h prior to induction, at induction time, and 1 h after induction time. The fermentation run was terminated after 19.97 h of elapsed fermentation time, and 4.97 h after the induction time.

TABLE 5
Fermentation Operating Conditions

| Condition | Initial | | Minimum | | Maximum | |
|---|---|---|---|---|---|---|
| Stirring | 220 | rpm | 220 | rpm | 1200 | rpm |
| Air Flow | 3 | SLPM | 3 | SLPM | 30 | SLPM |
| Temperature | 37° | C. | 37° | C. | 37° | C. |
| pH | 6.7 | | 6.7 | | 6.7 | |
| Pressure | 0.500 | atm | 0.500 | atm | 0.500 | atm |
| | (50.7 | kPa) | (50.7 | kPa) | (50.7 | kPa) |

| | | | |
|---|---|---|---|
| Dissolved O₂* | 20% | 20% | 20% |

*Cascade stirrer, then air flow.

TABLE 6
Composition of Feed Medium

| Component | Concentration | |
|---|---|---|
| MgSO₄•7H₂O | 2.0 | g/L |
| Glucose | 500 | g/L |
| Ampicillin | 150 | mg/L |
| (NH₄)₂HPO₄ | 4 | g/L |
| KH₂PO₄ | 9 | g/L |
| Yeast extract | 5.0 | g/L |
| Trace Elements - Feed (Table 7) | 10 | mL/L |

TABLE 7
Trace Elements - Feed

| Component | Concentration, mg/L |
|---|---|
| EDTA | 1300 |
| CoCl₂•H₂O | 400 |
| MnCl₂•4H₂O | 2350 |
| CuCl₂•2H₂O | 250 |
| H₃BO₃ | 500 |
| Na₂MoO₄•2H₂O | 400 |
| Zn(CH₃COO)₂•H₂O | 1600 |
| Ferric citrate | 4000 |

### Isolation and Purification of Peptide:

After completion of the fermentation run, the entire fermentation broth was passed three times through an APV model 2000 Gaulin type homogenizer at 12,000 psi (82,700 kPa). The broth was cooled to below 5° C. prior to each homogenization. The homogenized broth was immediately processed through a Westfalia WHISPERFUGE™ (Westfalia Separator Inc., Northvale, N.J.) stacked disc centrifuge at 700 mL/min and 12,000 RCF to separate inclusion bodies from suspended cell debris and dissolved impurities. The recovered paste was re-suspended at 15 g/L (dry basis) in water and the pH was adjusted to a value between 8.0 and 10.0 using Na₂CO₃/NaOH buffer. The pH was chosen to help remove cell debris from the inclusion bodies without dissolving the inclusion body proteins. The suspension was passed through the APV 2000 Gaulin type homogenizer at 12,000 psi (82,700 kPa) for a single pass to provide rigorous mixing. The homogenized high pH suspension was immediately processed in a Westfalia WHISPERFUGE™ stacked disc centrifuge at 700 mL/min and 12,000 RCF to separate the washed inclusion bodies from suspended cell debris and dissolved impurities. The recovered paste was resuspended at 15 gm/L (dry basis) in pure water. The suspension was passed through the APV 2000 Gaulin type homogenizer at 12,000 psi (82,700 kPa) for a single pass to provide rigorous washing. The homogenized suspension was immediately processed in a Westfalia WHISPERFUGE™ stacked disc centrifuge at 700 mL/min and 12,000 RCF to separate the washed inclusion bodies from residual suspended cell debris and NaOH.

The recovered paste was resuspended in pure water at 25 g/L (dry basis) and the pH of the mixture was adjusted to 2.2 using HCl. The acidified suspension was heated to 70° C. for 5 to 14 h to complete cleavage of the DP site separating the fusion peptide from the product peptide without damaging the target peptide. The product slurry was adjusted to pH 5.1 (note: the pH used here may vary depending on the solubility of the peptide being recovered) using NaOH and then was cooled to 5° C. and held for 12 h. The mixture was centrifuged at 9000 RCF for 30 min and the supernatant was decanted. The supernatant was then filtered with a 0.45 μm membrane. For some low solubility peptides, multiple washes of the pellet may be required to increase peptide recovery.

The filtered product was collected and concentrated by vacuum evaporation by a factor of 2:1 before lyophilization. Spectrophotometric detection at 220 and 278 nm was used to monitor and track elution of the product peptide.

### Preparation of Peptide-Based Hair Protectant

Acyl chloride functionalized cinnamic acid (3-phenyl-2-propenoyl chloride, CAS No. 102-92-1, obtained from Aldrich; Milwaukee, Wis.), 11.2 mg, was dissolved in 3 mL of 1-methyl-2-pyrrolidone (NMP) and added to a solution containing 100 mg of the multi-block hair-binding peptide (SEQ ID NO:33), having associated trifluoroacetate counter-ions, dissolved in 20 mL of NMP containing triethylamine (50 mg). The resulting solution was stirred at room temperature for 72 h. The solvent was removed by evaporation, yielding 108 mg of a light brown crystalline solid.

The product was analyzed by gas chromatography-MALDI mass spectrometry and found to have a molecular weight distribution consistent with covalent attachment of multiple cinnamic acid moieties to the peptide. Unreacted peptide and peptide fragments were also present in the reaction mixture in small amounts.

### Example 3

### Prophetic

### Shampoo Composition Comprising a Peptide-Based Hair Protectant

The purpose of this prophetic Example is to describe how to prepare a shampoo composition comprising a peptide-based hair protectant.

The shampoo composition is prepared using the ingredients listed in Table 8.

TABLE 8
Shampoo Composition

| Ingredient | Wt % |
|---|---|
| Ammonium laureth sulfate | 12 |
| Sodium laureth sulfate | 5 |
| Di(hydrogenated) tallow phthalic acid | 4 |
| amide | |
| Cocamide MEA | 2 |
| Polyquaternium-10 | 1 |
| Peptide-based hair protectant as | 7.5 |
| described in Example 1 or 2 | |
| Citric acid | to adjust pH |
| Disodium EDTA | 0.5 |
| Fragrance | 0.7 |
| Water | qs to 100 |

The shampoo composition is prepared by combining water and the EDTA, heating to 65° C. and mixing until the EDTA is dissolved. Then the remaining ingredients are added, and the mixture is mixed until all the solids are dissolved and the color is uniform. The pH is adjusted with citric acid as desired.

### Example 4

### Prophetic

### Hair Conditioner Composition Comprising a Peptide-Based Hair Protectant

The purpose of prophetic Example is to describe how to prepare a hair conditioner composition comprising a peptide-based hair protectant.

A hair conditioner is prepared by mixing the ingredients listed in Table 9.

TABLE 9
Hair Conditioner Composition

| CFTA Names | wt % |
|---|---|
| Self emulsifying glyceryl fatty acid ester | 6.0 |
| Cetrimonium chloride | 3.5 |
| Dicetyldimonium chloride | 3.0 |
| Cetearyl alcohol | 2.0 |
| Peptide-based hair protectant as described in Example | 10.0 |
| 1 or 2 | |
| Trimethylsilylamodimethicone | 0.7 |
| Menthol | 0.1 |
| Phytolipid and hyaluronic acid | 0.1 |
| Apricot seed (Apricot Kernel Powder produced by | 0.25 |
| Alban Muellen, Inc. of Paris, France) | |
| Pearlizing agent | 0.8 |
| Methyl gluceth-20 | 0.25 |
| Polyquaternium-4 | 0.1 |
| Water | qs to 100 |

To 55 g of deionized water heated to 60° C., the first 4 ingredients are added serially with moderate agitation until completely dissolved. The bulk solution is then cooled to 35° C., and the remaining ingredients are added serially with moderate agitation.

What is claimed is:

1. A peptide-based hair protectant having the general structure:
(HBPₘ)ₙ-(SCA)_{y},
or
[(HBP)ₚ-S_{q}]ₙ-(SCA)_{y}
wherein
a) HBP is a hair-binding peptide;
b) SCA is a sunscreen agent;
c) m ranges from 1 to about 100;
d) n ranges from 1 to about 100;
e) y ranges from 1 to about 100;
f) S is a spacer;
g) p ranges from 1 to 10; and
h) q ranges from 1 to about 100.

2. The peptide-based hair protectant according to claim 1 wherein the hair-binding peptide is from about 7 to about 50 amino acids in length.

3. The peptide-based hair protectant according to claim 1 wherein the hair-binding peptide is generated combinatorially by a process selected from the group consisting of phage display, yeast display, ribosome display, mRNA display, and bacterial display.

4. The peptide-based hair protectant according to claim 1 wherein the hair-binding peptide is selected from the group consisting of SEQ ID NOs: 1-28, 33, and 42-58.

5. The peptide-based hair protectant according to claim 1 wherein the hair-binding peptide further comprises at least one cysteine residue on at least one end of the peptide selected from the group consisting of
a) the N-terminal end; and
b) the C-terminal end.

6. The peptide-based hair protectant according to claim 1 wherein the hair-binding peptide further comprises at least one lysine residue on at least one end of the peptide selected from the group consisting of
a) the N-terminal end; and
b) the C-terminal end.

7. The peptide-based hair protectant according to claim 1 wherein the sunscreen agent is selected from the group consisting of: oxides of titanium, zinc, cerium, or iron; titanium dioxide nanoparticles, para-aminobenzoic acid, ethyl para-aminobenzoate, amyl para-aminobenzoate, octyl para-aminobenzoate, ethylhexyl dimethyl para-aminobenzoate, ethylene glycol salicylate, phenyl salicylate, octyl salicylate, benzyl salicylate, butylphenyl salicylate, homomethyl salicylate, ethylhexyl salicylate, triethanolamine salicylate, benzyl cinnamate, 2-ethoxyethyl para-methoxycinnamate, ethylhexyl methoxycinnamate, octyl para-methoxycinnamate, glyceryl mono(2-ethylhexanoate) di-para-methoxycinnamate, isopropyl para-methoxycinnamate, urocanic acid, ethyl urocanate, hydroxymethoxybenzophenone, hydroxymethoxybenzophenonesulfonic acid, hydroxymethoxybenzophenonesulfonic acid salts, dihydroxymethoxybenzophenone, sodium dihydroxymethoxybenzophenonedisulfonate, dihydroxybenzophenone, tetrahydroxybenzophenone, 4-tert-butyl-4′-methoxydibenzoylmethane, phenylbenzimidazole sulfonic acid, 2,4,6-trianilino-p-(carbo-2′-ethylhexyl-1′-oxy)-1,3,5-triazine, octocrylene, menthyl anthranilate, 2-(2-hydroxy-5-methylphenyl)benzotriazole, avobenzone, cinnamic acid, and organic polymers that scatter ultraviolet radiation.

8. The peptide-based hair protectant according to claim 1 wherein the hair-binding peptide is identified by a process comprising the steps of:
(a) providing a combinatorial library of DNA associated peptides;
(b) contacting the library of (a) with a hair sample to form a reaction mixture comprising DNA associated peptide-hair complexes;
(c) isolating the DNA associated peptide-hair complexes of (b);
(d) amplifying the DNA encoding the peptide portion of the DNA associated peptide-hair complexes of (c); and
(e) sequencing the amplified DNA of (d) encoding a hair-binding peptide, wherein the hair-binding peptide is identified.

9. The peptide-based hair protectant according to claim 8 wherein after step (c):
(i) the DNA associated peptide-hair complexes are contacted with an eluting agent whereby a portion of DNA associated peptides are eluted from the hair and a portion of the DNA associated peptides remain complexed; and
(ii) the eluted or complexed DNA associated peptides of (i) are subjected to steps (d) and (e).

10. The peptide-based hair protectant according to claim 8 wherein the DNA encoding a hair-binding peptide is amplified by a process selected from the group consisting of:
a) amplifying DNA comprising a hair-binding peptide coding region by polymerase chain reaction; and
b) infecting a host cell with a phage comprising DNA encoding the hair-binding peptide and growing said host cell in a suitable growth medium.

11. The peptide-based hair protectant according to claim 8 wherein the peptides encoded by the amplified DNA of step (d) are contacted with a fresh hair sample and steps (b) through (d) are repeated one or more times.

12. The peptide-based hair protectant according to claim 1 wherein the spacer is a peptide spacer comprising amino acids selected from the group consisting of proline, lysine, glycine, alanine, serine, and mixtures thereof.

13. The peptide-based hair protectant according to claim 12 wherein the peptide spacer is from 2 to about 50 amino acids in length.

14. The peptide-based hair protectant according to claim 12 wherein the peptide spacer comprises an amino acid sequence selected from the group consisting of SEQ ID NOs: 30, 31, 32, 36, 37, 38, 39, 40, and 41.

15. The peptide-based hair protectant according to claim 1 wherein the spacer is selected from the group consisting of ethanolamine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, ethyl alkyl chains, propyl alkyl chains, hexyl alkyl chains, steryl alkyl chains, cetyl alkyl chains, and palmitoyl alkyl chains.

16. A hair care composition comprising an effective amount of at least one peptide-based hair protectant according to claim 1.

17. The hair care composition according to claim 16 wherein the composition is selected from the group consisting of a shampoo, a conditioner, a rinse, a lotion, an aerosol, a gel, a mousse, and a hair dye.

18. The hair care composition according to claim 16 wherein the composition further comprises at least one cosmetic additive or adjuvant selected from the group consisting of antioxidants, preserving agents, fillers, surfactants, fragrances, thickeners, wetting agents, anionic polymers, nonionic polymers, amphoteric polymers, dyes, and pigments.

19. A method for forming a protective layer of a peptide-based hair protectant on hair comprising applying the composition of claim 16 to the hair and allowing the formation of said protective layer.

20. A method for forming a protective layer on hair comprising the steps of:
(a) providing a hair care composition comprising a peptide-based hair protectant selected from the group consisting of:
(HBPₘ)ₙ-(SCA)_{y}; and i)
[(HBP)ₚ-S_{q}]ₙ-(SCA)_{y} ii)
wherein
1) HBP is a hair-binding peptide;
2) SCA is a sunscreen agent;
3) n ranges from 1 to about 100;
4) S is a spacer;
5) m ranges from 1 to about 100;
6) p ranges from 1 to about 10;
7) q ranges from 1 to about 100; and
8) y ranges from 1 to about 100;
and wherein the hair-binding peptide is selected by a method comprising the steps of:
(A) providing a combinatorial library of DNA associated peptides;
(B) contacting the library of (A) with a hair sample to form a reaction mixture comprising DNA associated peptide-hair complexes;
(C) isolating the DNA associated peptide-hair complexes of (B);
(D) amplifying the DNA encoding the peptide portion of the DNA associated peptide-hair complexes of (C); and
(E) sequencing the amplified DNA of (D) encoding a hair-binding peptide, wherein the hair-binding peptide is identified; and
(b) applying the hair care composition of (a) to hair and allowing the formation of said protective layer.

21. The method according to claim 20 wherein after step (C):
(i) the DNA associated peptide-hair complexes are contacted with an eluting agent whereby a portion of DNA associated peptides are eluted from the hair and a portion of the DNA associated peptides remain complexed; and
(ii) the eluted or complexed DNA associated peptides of (i) are subjected to steps (D) and (E).

22. The method according to claim 20 wherein the DNA encoding a hair-binding peptide is amplified by a process selected from the group consisting of:
a) amplifying DNA comprising a hair-binding peptide coding region by polymerase chain reaction; and
b) infecting a host cell with a phage comprising DNA encoding the hair-binding peptide and growing said host cell in a suitable growth medium.

23. The method according to claim 20 wherein the peptides encoded by the amplified DNA of step (D) are contacted with a fresh hair sample and steps (B) through (D) are repeated one or more times.
